(19) United States
(12) Patent Application Publication
(10) Pub. No.: US 2008/0051393 A1
(43) Pub. Date: Feb. 28, 2008

(54) Novel cannabinoids and methods of use

(75) Inventors: Patricia B. Williams, Norfolk, VA (US); Billy R. Martin, Richmond, VA (US); Frank A. Lattanzio, Chesapeake, VA (US); Sandeep Samudre, Norfolk, VA (US); Raj K. Razdan, St. Woburn, MA (US)

Correspondence Address: WILMERHALE/DC, 1875 PENNSYLVANIA AVE., NW, WASHINGTON, DC, 20004, US

(73) Assignee: Eastern Virginia Medical School, Norfolk, VA (US)

(21) Appl. No.: 11/797,223

(22) Filed: May 1, 2007

Related U.S. Application Data
(60) Provisional application No. 60/796,143, filed on May 1, 2006.

(51) Int. Cl. A61K 31/40; A61K 31/41; A61K 31/415; A61K 31/445; A61K 31/535; A61P 43/00; C07D 211/20; C07D 231/10; C07D 265/30

(52) U.S. Cl. 514/232.8; 514/237.8; 514/315; 514/359; 514/383; 514/397; 514/399; 514/406; 514/428; 544/175; 546/248; 548/268.6; 548/376.1

(57) ABSTRACT

Water- and lipid-soluble cannabinoid compounds that are agonists for CB1 and CB2 receptors are provided. The cannabinoid compounds are useful for the treatment of retinal neurodegenerative disorders such as glaucoma, diabetic retinopathy and macular degeneration.

### FIELD OF THE INVENTION

Embodiments of the present invention generally relate to novel cannabinoid compounds and methods of using them. In particular, the invention provides water-soluble and lipid-soluble cannabinoid compounds that are agonists of CB 1 and CB2 receptors, and that are useful for treating or alleviating retinal neurodegenerative disorders or symptoms thereof, including, for example, glaucoma, diabetic retinopathy and macular degeneration. Embodiments of the present invention may be used to reduce intraocular pressure (IOP) or peripheral retinal ganglion cell loss associated with retinal neurodegenerative disorders. Embodiments of the present invention may also be used to administer these neuroprotective cannabinoid agonists alone or in combination with other agents.

### BACKGROUND OF THE INVENTION

Glaucoma is the leading cause of preventable blindness in the United Sates and the second leading cause of blindness in the world. Glaucoma can affect people of all ages, but it is more common in adults over the age of 65, in certain ethnic groups, in people diagnosed with diabetes and/or myopia. Although there are various different types of glaucoma, the common features that characterize glaucoma are elevated intraocular pressure (IOP) associated with optic cupping and visual field loss.

The loss of vision experienced by glaucoma patients is a result of retinal damage and is most directly caused by diffuse retinal ganglion cell (RGC) atrophy. Loss of ganglion cell axons leads to axonal loss in the optic nerve. If left untreated, glaucoma can result in significant damage to the retina and optic nerve, leading to complete blindness in the affected eye(s). In general, the higher the IOP, the more likely the loss of visual function. A 30-40% decrease in high IOP reduces the risk of developing glaucoma by over 65%. Reducing the risk slows the progression of glaucoma but is not indicative of retinal health. Current pharmacologic and surgical treatments for glaucoma primarily focus on reducing IOP. Though current pharmacological treatments have been successful in reducing IOP in many people suffering from glaucoma, the potential side effects from these medications cannot be ignored and must be carefully taken into consideration for each individual patient. Half of all glaucoma patients cannot be maintained on single drug therapy e.g., timolol alone, and most require use of two or even three drugs to control their IOP. In addition, some patients do not respond well to lowering of IOP.

Current therapy for managing high IOP for glaucoma is limited to adrenergic agonists. These adrenergic drugs decrease IOP but have a short duration. Cannabinoids modulate IOP and work by activating the CB receptor system. They are potent hypotensive agents and their effects last for at least two hours, therefore they may be administered at lower concentrations than their counterparts. This is a major advantage because only a small amount of drug is needed to achieve clinically significant effects on IOP, and because of their long lasting effects, the topical administration regimen may also be reduced. Targeting the CB receptors instead of the adrenergic system may solve the problem of efficacy and durability. Great progress has been made in the last fifteen years in understanding the mechanisms by which cannabinoids work. Two cannabinoid I receptors have been sequenced, CB1 receptors, which are localized in the CNS and periphery, and CB2 receptors, which have been identified only in the periphery. The CB system plays a physiological role in pain perception, cognitive processes, neurotransmitter regulation, appetite control, regulation of IOP, and reward system, to name just a few. The development of SR 141716A, an antagonist that is selective for CBI receptors, and SR 144528, an antagonist selective for the CB2 receptor, were important tools to elucidate receptor mechanisms of action. CB1 cannabinoid receptors have been found in the rat eye in toto and specifically in the ciliary body and iris using RT-PCR. The discovery of several endogenous cannabinoids, including arachidonylethanolamide (anandamide), 2 arachidonoylglycerol (2 AG), and palmitoylethanolamide (PEA), suggest the existence of an endogenous cannabinoid system.

Moreover, blindness or additional visual field loss actually continues to occur in approximately 25 to 38% of glaucoma patients, despite controlled treatment of IOP. Glaucomatous damage to the retina and optic nerve progresses even after therapy to maintain normal IOP. Since glaucoma often continues to progress, even with good patient compliance and careful control of IOP, many agree that other factors may be involved. Neuroprotective agents help optic nerve survival by preventing damage to nerve fibers or by enhancing the survival of existing retinal ganglion cells. Compared to untreated rats, rats treated with aminoguanidine, a noncanabinoid neuroprotectant, had 26% less peripheral retinal ganglion cell loss. Thus, neuroprotective agents could have a very large potential in the therapy and treatment of glaucoma patients.

Cannabis, or marijuana, has been studied extensively for medicinal use since the 1970s. However, controversy continues over the use of marijuana in medicine due to the various undesirable systemic side effects that are associated with marijuana inhalation. Because glaucoma is a neurodegenerative disease, an ideal treatment should not only reduce IOP, but enhance the survival of the optic nerve as well. The most extensively studied cannabinoid is the active ingredient in marijuana, Δ9-tetrahydrocannabinol (THC). The effect of vehicle on the transcorneal flux of THC has been measured on isolated rabbit corneas (Kearse, 2000) and the ability of THC to cross the cat cornea was also demonstrated (Green, 1977). Unlike THC, the novel cannabinoids described herein represent a new class of cannabinomimetics and have a structure that is different from that of other natural cannabinoids. These drugs, hereby classified as novel cannabinoids, are potent cannabinoid receptor (CB) agonists. The novel cannabinoids described herein not only achieve the therapeutic benchmark in IOP reduction, but have also been suprisingly shown to protect the retina, specifically retinal ganglionic cells (RGC). This neuroprotective effect is a significant advantage held by these novel cannabinoids over past solutions to treat glaucoma.

### SUMMARY OF THE INVENTION

The compounds of the invention are useful for the treatment of potential blinding and eye damaging diseases, such as glaucoma, diabetic retinopathy and macular degeneration. These conditions are often associated with degeneration of the retina and optic nerve. Elevated IOP is a major risk factor for glaucoma. There is also a significant number of glaucoma patients with normal IOP but increasing retinal damage. This argues for a direct component in glaucoma resulting in damage to the retina and optic nerve that is independent of IOP. It has been discovered that the cannabinoids described herein provide unexpected beneficial results in reducing IOP and/or protecting the retina.

The compounds of the invention include potent cannabinoid receptor (CB) agonists. They have the surprising and unexpected ability to protect the retina that is independent of their effects to decrease IOP. Following topical administration to the eye, they do not exhibit the systemic adverse effects. This is important because topical application may bypass many systemic side effects that have frustrated earlier attempts to develop cannabinoids to decrease IOP.

In one embodiment, the invention provides a compound, and pharmaceutically acceptable salts thereof, having the formula (I): 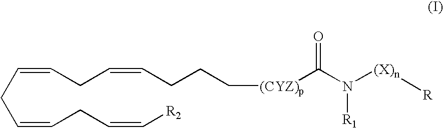
wherein

R is CH₃, OH, Cl, F, Br, I, or CF₃;

R₁ is H, or C₁-C₆ alkyl;

X is alkyl, branched alkyl, cyclopropyl, or cycloalkyl;

Y is H, alkyl, or branched alkyl;

Z is H, alkyl, or branched alkyl;

p is 0-4; and

R₂ is 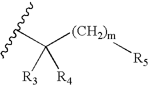
wherein
   R₃ is H, alkyl, or branched alkyl;
   R₄ is H, alkyl, or branched alkyl;
   m is 1-7; and
   R₅ is CH₃, OH, Cl, Br, I, CN, ONO, ONO₂, NO₂, pyrazole, triazole, morpholine, piperidine, pyrrolidine, or imidazole;
provided that when R is OH, R₁ is H, X is isopropyl, p is 0, R₃ is methyl, R₄ is methyl, m is 4, R₅ cannot be CN.

In another embodiment, the compounds are selected from the following:

| Compound | Structure |
|---|---|
| O-2954 | 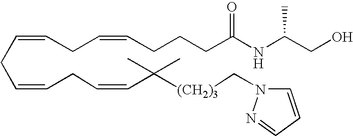 |
| O-2955 | 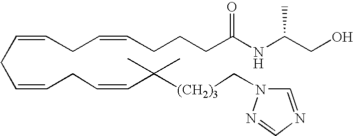 |
| O-4316 | 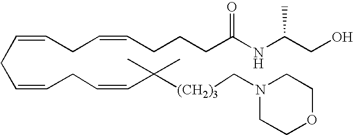 |
| O-4368 | 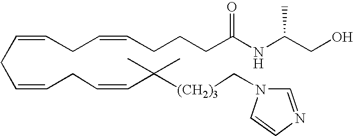 |
| O-4386 | 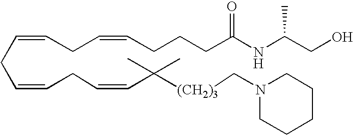 |

In another embodiment, the invention provides for a method for protecting a patient against a disorder characterized by damage to the retina or optic nerve, comprising administering to a patient in need thereof an effective amount of a compound, or pharmaceutically acceptable salt thereof, having the formula (I): 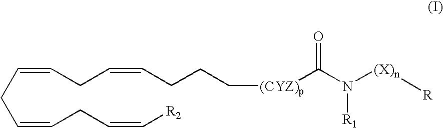
wherein

R is CH₃, OH, Cl, F, Br, I, or CF₃;

R₁ is H, or C₁-C₆ alkyl;

X is alkyl, branched alkyl, cyclopropyl, or cycloalkyl;

Y is H, alkyl, or branched alkyl;

Z is H, alkyl, or branched alkyl;

p is 0-4; and

R₂ is 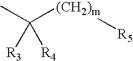
wherein
   R₃ is H, alkyl, or branched alkyl;
   R₄ is H, alkyl, or branched alkyl;
   m is 1-7; and
   R₅ is CH₃, OH, Cl, Br, I, CN, ONO, ONO₂, NO₂, pyrazole, triazole, morpholine, piperidine, pyrrolidine, or imidazole.

In another embodiment, the invention provides for a method for protecting a patient against a disorder characterized by damage to the retina or optic nerve, comprising administering to a patient in need thereof an effective amount of a compound selected from the following:

| Compound | Structure |
|---|---|
| O-2954 | 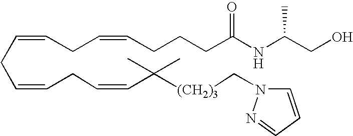 |
| O-2955 | 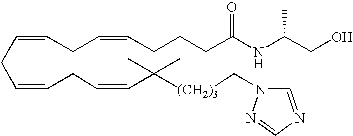 |
| O-4316 | 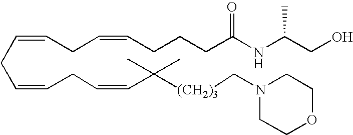 |
| O-4368 | 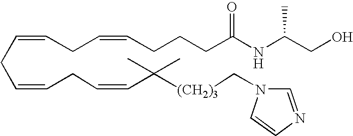 |
| O-4386 | 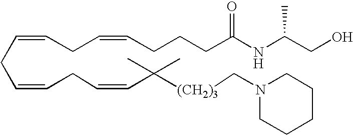 |
| O-4388 | 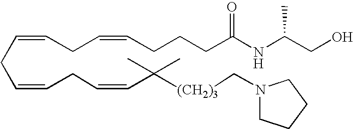 |
| O-1812 | 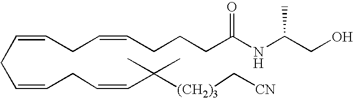 |

In another embodiment, the invention provides a method for protecting a patient against a disorder characterized by damage to the retina or optic nerve, comprising administering to a patient in need thereof an effective amount of a compound, or pharmaceutically acceptable salt thereof, having the formula (II): 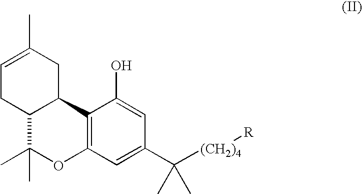
wherein R is imidazole, pyrazole, triazole, or morpholine.

In another embodiment, the invention provides a method for protecting a patient against a disorder characterized by damage to the retina or optic nerve, comprising administering to a patient in need thereof an effective amount of a compound, or pharmaceutically acceptable salt thereof, selected from the following:

| Compound | Structure |
|---|---|
| O-2545 | 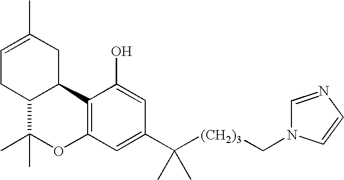 |
| O-2651 | 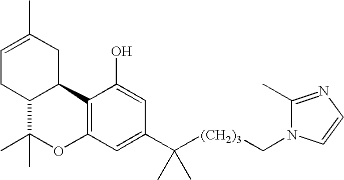 |
| O-2715 | 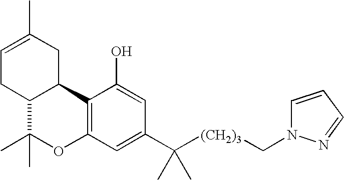 |
| O-2716 | 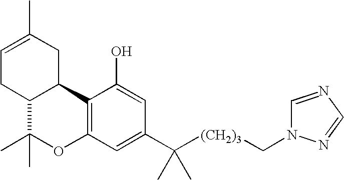 |
| O-2737 | 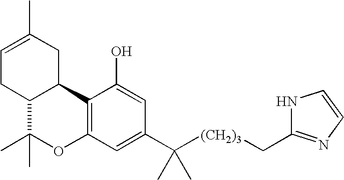 |
| O-3226 | 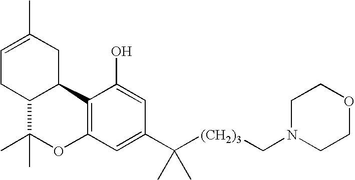 |

In another embodiment, the invention provides a method for protecting retinal ganglionic cells, comprising administering an effective amount of a compound, or pharmaceutically acceptable salt thereof, having the formula (1): 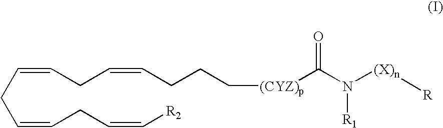
wherein

R is CH₃, OH, Cl, F, Br, I, or CF₃;

R₁ is H, or C₁-C₆ alkyl;

X is alkyl, branched alkyl, cyclopropyl, or cycloalkyl;

Y is H, alkyl, or branched alkyl;

Z is H, alkyl, or branched alkyl;

p is 0-4; and

R₂ is 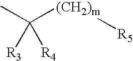
wherein
   R₃ is H, alkyl, or branched alkyl;
   R₄ is H, alkyl, or branched alkyl;
   m is 1-7; and
   R₅ is CH₃, OH, Cl, Br, I, CN, ONO, ONO₂, NO₂, pyrazole, triazole, morpholine, piperidine, pyrrolidine, or imidazole.

In another embodiment, the invention provides a method for protecting retinal ganglionic cells, comprising administering an effective amount of a compound, or pharmaceutically acceptable salt thereof, wherein compound is selected from the following:

| Compound | Structure |
|---|---|
| O-2954 | 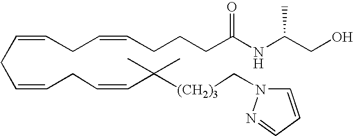 |
| O-2955 | 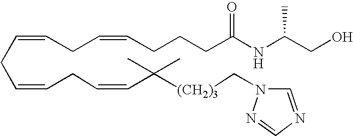 |
| O-4316 | 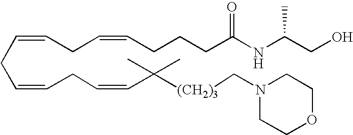 |
| O-4368 | 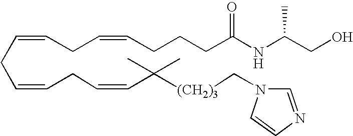 |
| O-4386 | 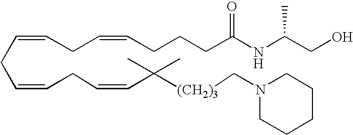 |
| O-4388 | 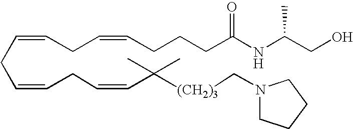 |
| O-1812 | 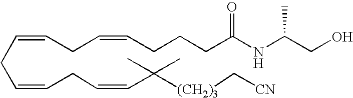 |

In another embodiment, the invention provides a method for protecting retinal ganglionic cells, comprising administering an effective amount of a compound, or pharmaceutically acceptable salt thereof, having the formula (II): 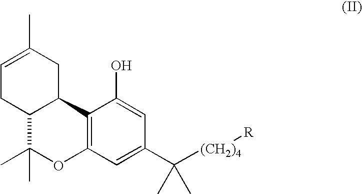
wherein R is imidazole, pyrazole, triazole, or morpholine.

In another embodiment, the invention provides a method for protecting retinal ganglionic cells, comprising administering an effective amount of a compound, or pharmaceutically acceptable salt thereof, wherein the compound is selected from the following:

| Compound | Structure |
|---|---|
| O-2545 | 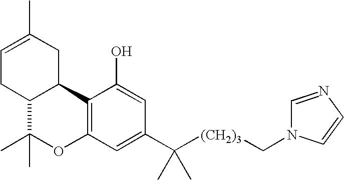 |
| O-2651 | 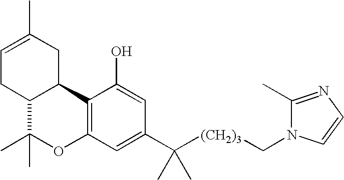 |
| O-2715 | 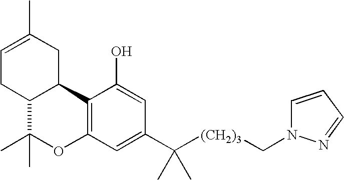 |
| O-2716 | 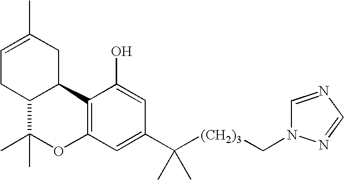 |
| O-2737 | 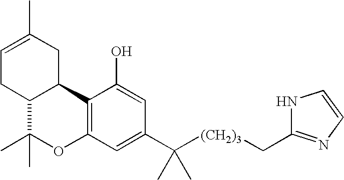 |
| O-3226 | 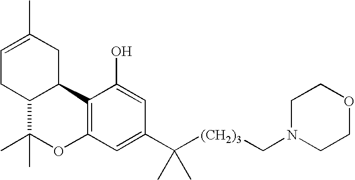 |

In another embodiment, the invention provides a method for protecting retinal ganglionic cells, wherein said retinal ganglionic cells are in vivo.

### DESCRIPTION OF THE FIGURES

FIG. 1. O-1812 Intraocular Pressure (IOP) Profile. When administered topically, all concentrations of O-1812 (anandamide analog) significantly reduced IOP compared to baseline or vehicle (n=5). 0.01% O-1812 also significantly reduced IOP, but this decrease was not maintained beyond 60 min (n=5). 1.0% O-1812 was most efficacious.

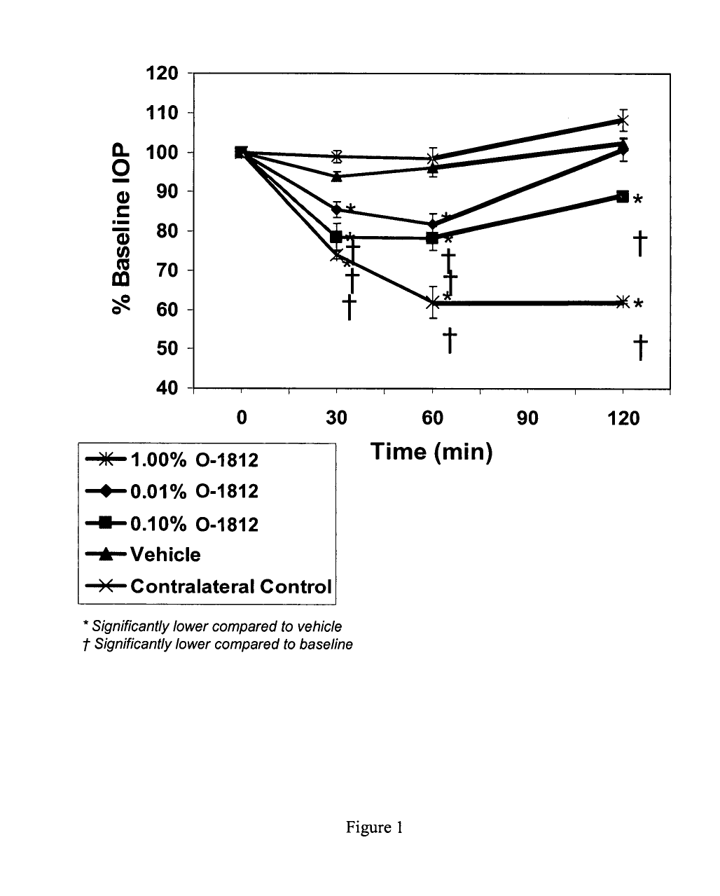

FIG. 2. O-2545 IOP Profile. At 30 min, both 0.1% and 1.0 % O-2545 (THC analog) significantly reduced IOP (p=0.03 and 0.05 respectively) compared to vehicle, (n=7). When compared to baseline or vehicle, 0.1% and 1.0% significantly reduced IOP up to 120 min (p<0.05, n=7).

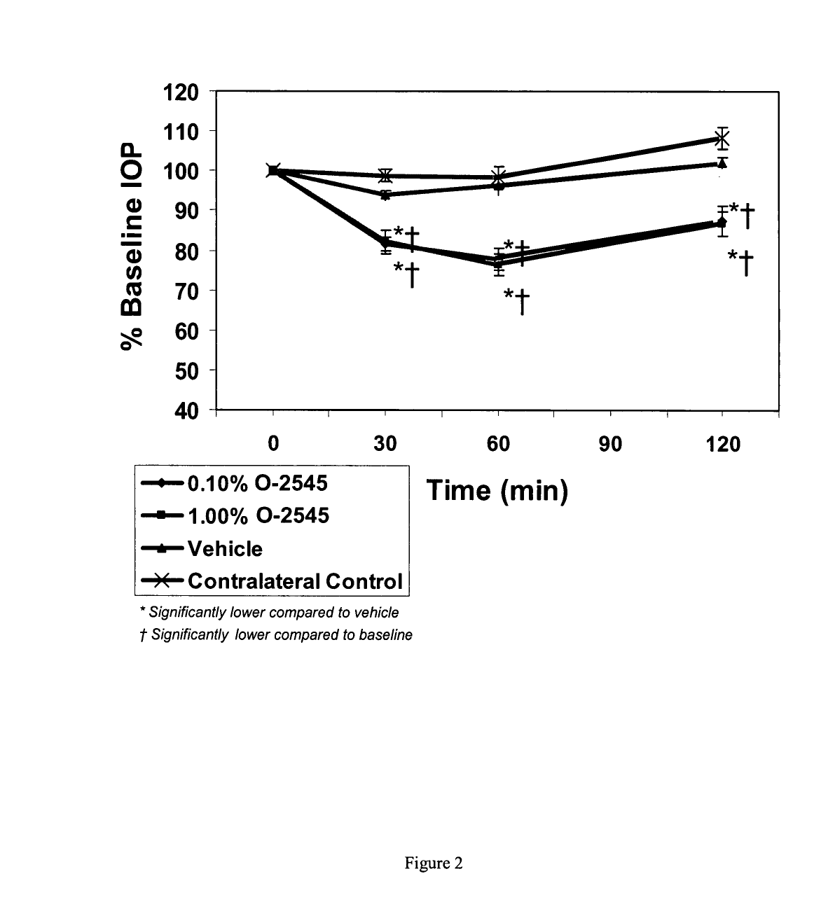

FIG. 3. WIN 55-212-3 IOP Profile. WIN 55-212-3 is a less active enantiomer of WIN 55-212-2. At 30 min, IOP was significantly lowered compared to the baseline or vehicle (p<0.01, n=8). This decrease in IOP was maintained for up to 120 min (p<0.01, n=8).

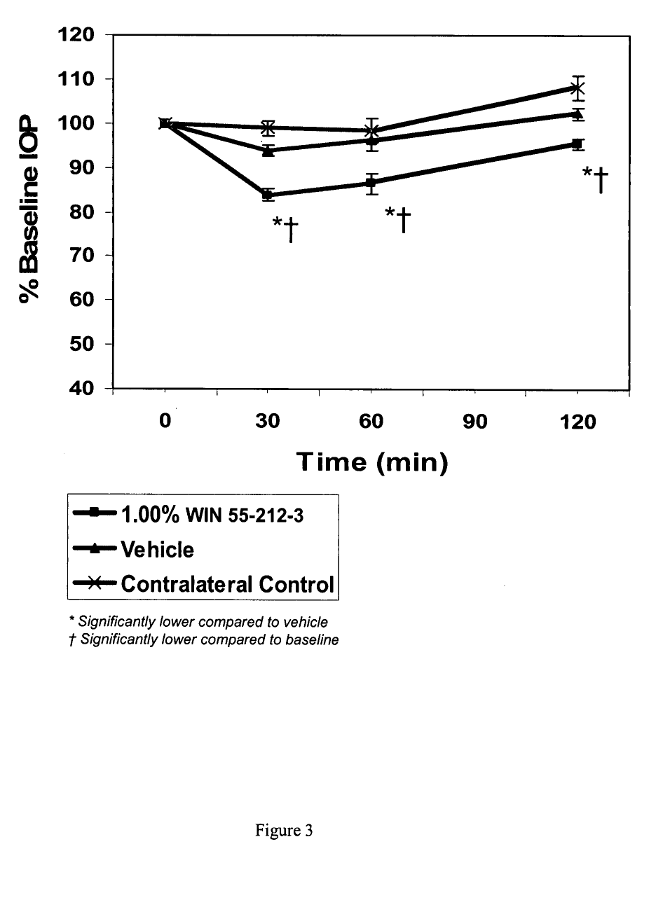

FIG. 4. WIN 55-212-2 IOP Profile. At 30 min, only 1.0% significantly reduced IOP compared to baseline (p=0.01, n=11). At 60 min, 1.0% was significantly reduced when compared to baseline (p<0.01, n=11) or vehicle (p=0.01, n=11). This reduction in IOP was maintained up to 120 min (p<0.01, n=11). Although 0.1% reduced IOP, the decrease was not significantly different from either vehicle or baseline (p=0.11, n=4).

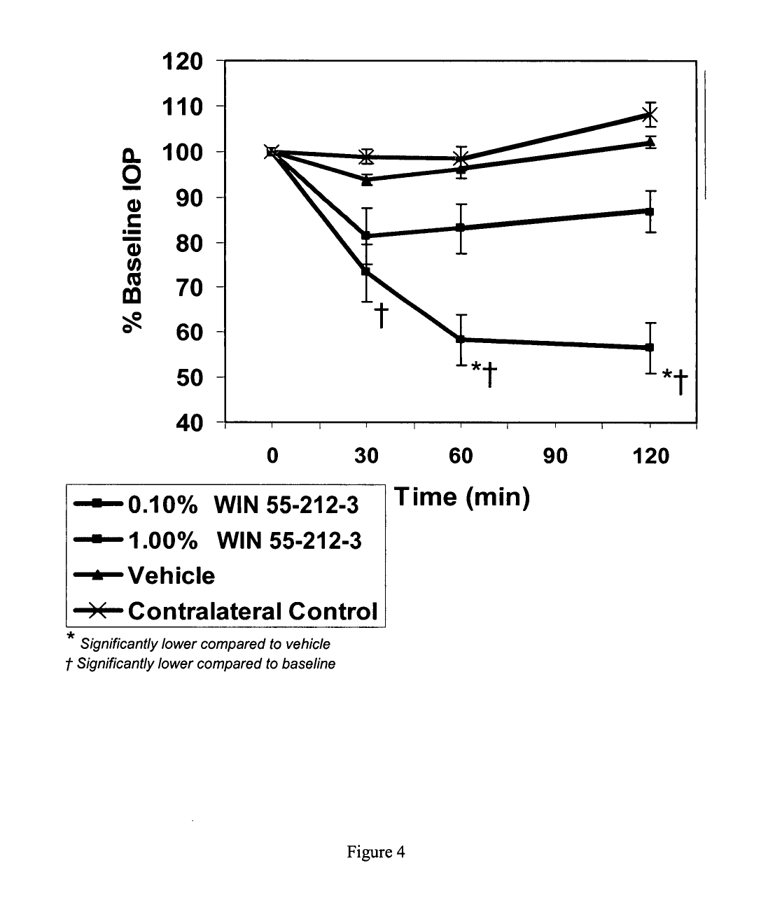

FIG. 5. Systemic Effects. Following topical application, no drug affected heart rate (HR), systolic or diastolic blood pressure compared to baseline (p>0.6, n=5).

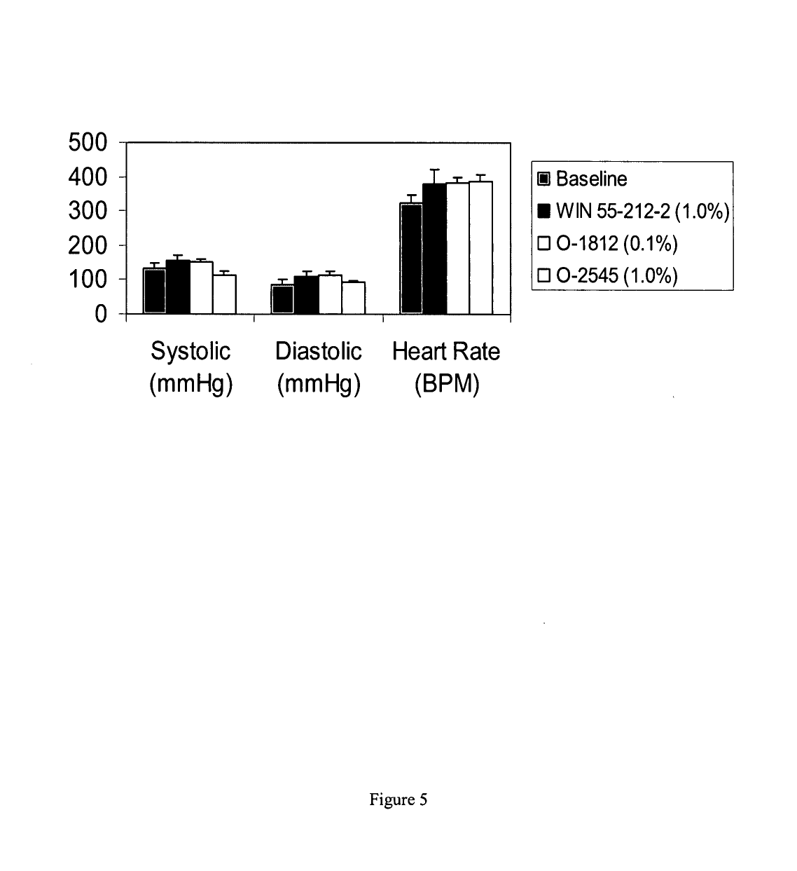

FIG. 6. Corneal thickness measured by in vivo confocal microscopy. Corneal thickness was measured in eyes treated with WIN 55-212-2 1.0% (OD) and contralateral control (OS). Corneal thickness remained unchanged before treatment (p=0.321) and after treatment (p=0.263).

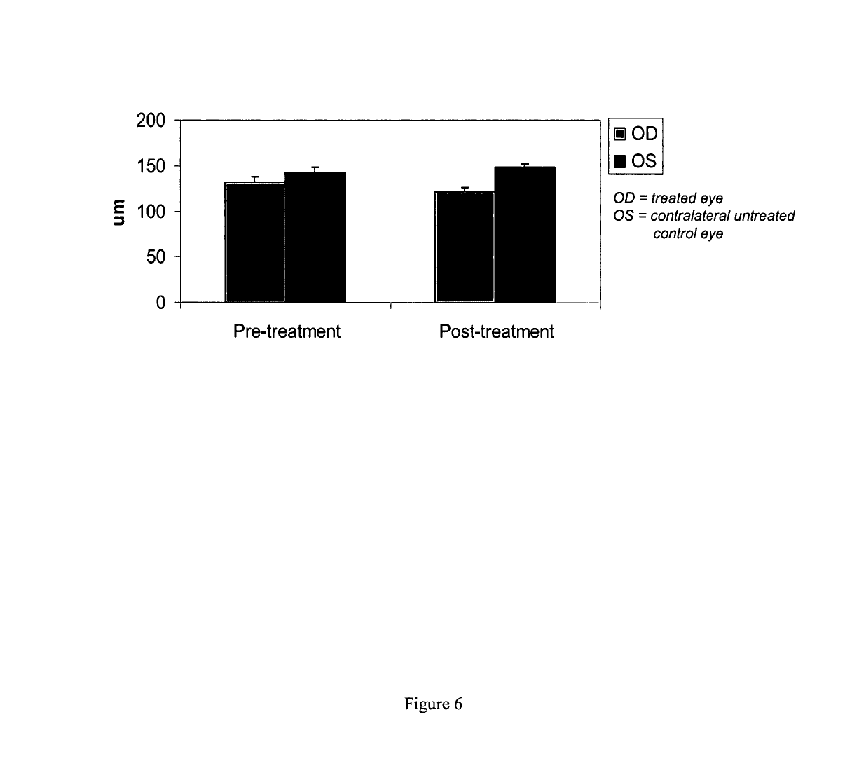

FIG. 7. Neuroprotection (A-Wave ERG Profile). Novel cannabinoids protect the retinal function. O-1812, a lipophilic and CB1 agonist, more effectively preserved retinal function. Data is presented as a percentage of the contralateral normal eye (OS). (OD), treated eye.

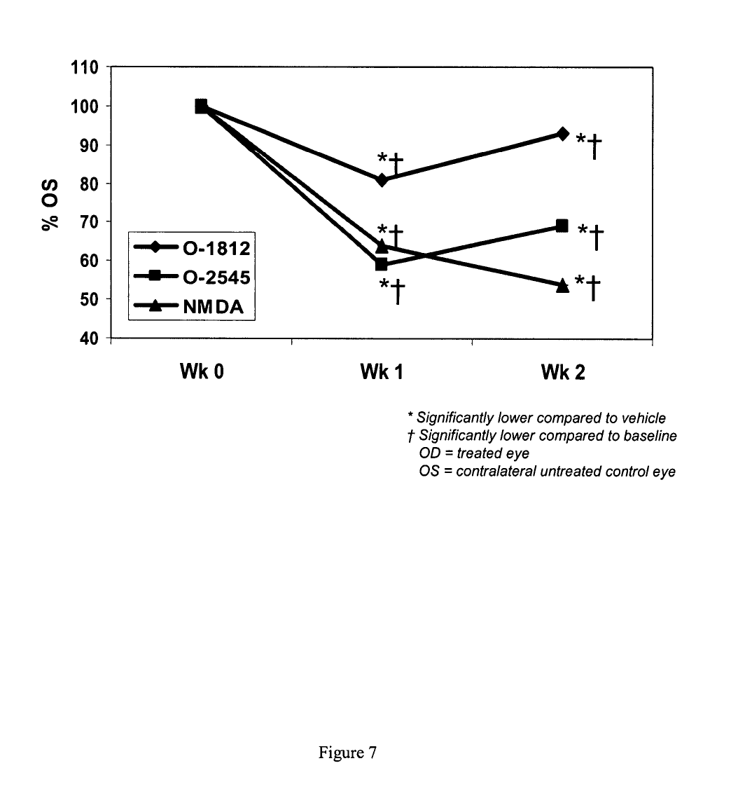

FIG. 8. Effect of CB1 Antagonist SR-141716 (ERG A-Wave Profile). CB1 antagonist effectively blocked the action of both O-1812 and O-2545. A significant functional a-wave loss was seen at week 1. Error bars are not shown because they are <0.5% of the mean.

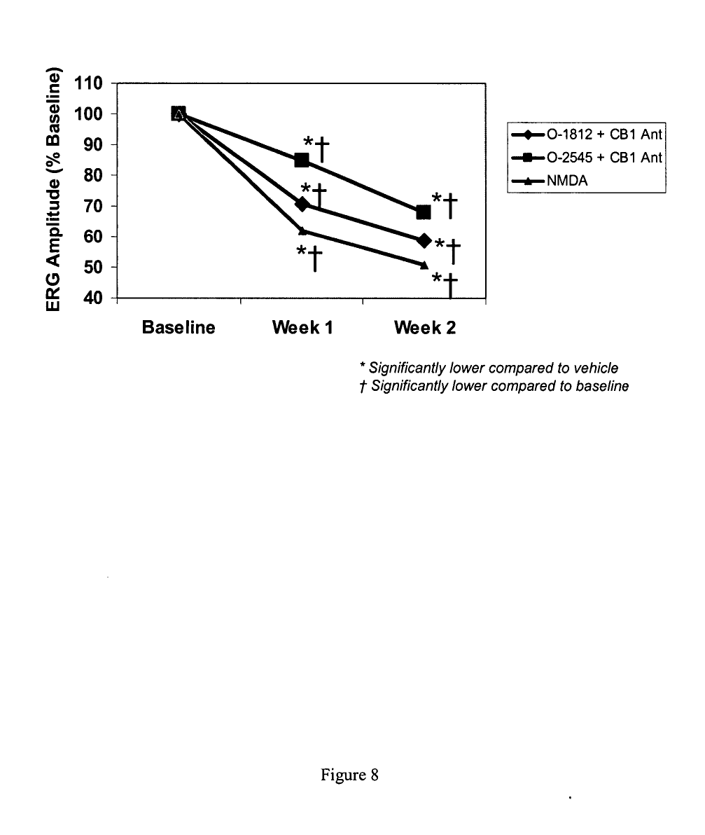

FIG. 9. Effect of CB2 Antagonist SR-144258 (ERG A-Wave Profile). CB2 antagonist had no effect on the action of O-1812. The action of O-2545 was affected by the presence of CB2 antagonist. NMDA only treatment resulted in significant functional loss. Error bars are not shown because they are <0.5% of the mean.

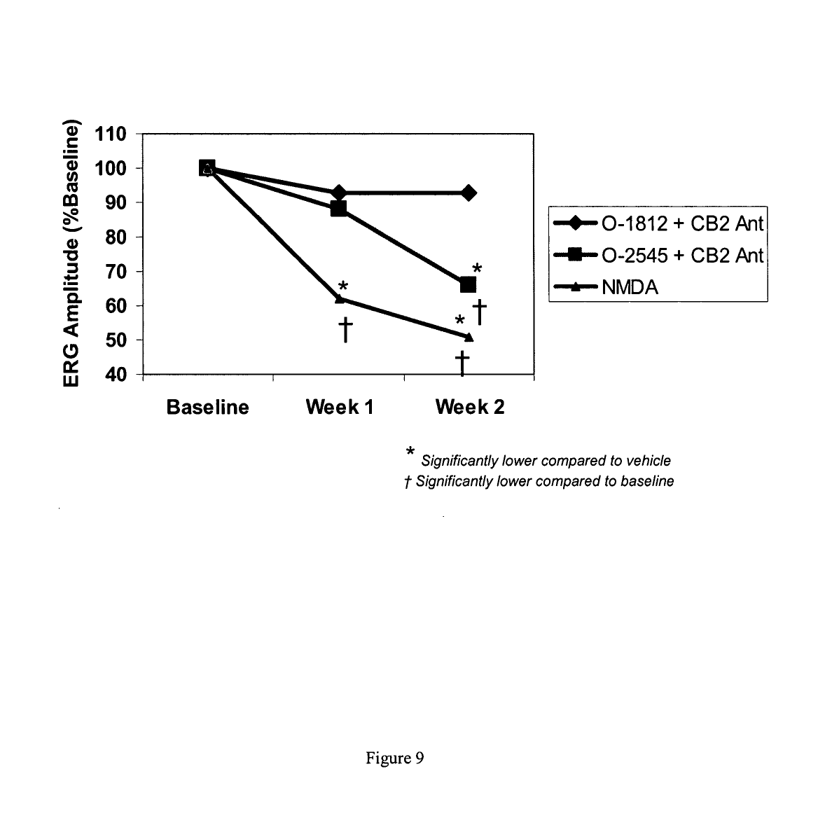

FIG. 10. Retinal Flat Mounts of Retinal Ganglionic Cells. NMDA treatment was toxic to retinal ganglionic cells (RGC); cell density was reduced by 49%. Both O-1812 and O-2545 preserved the number of RGCs. O-1812 treatment preserved up to 83% of RGCs. All 40× magnifications.

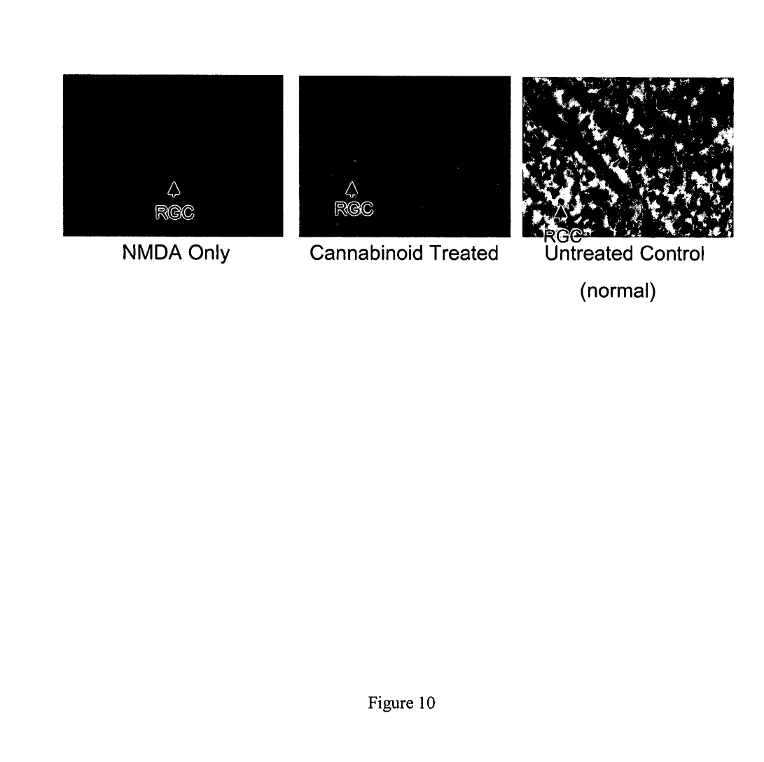

FIG. 11. Optic Nerve. NMDA treatment damaged the optic nerve. This damage reduced the diameter of the optic nerve. Both O-1812 and O-2545 treatment prevented much of the NMDA induced damage.

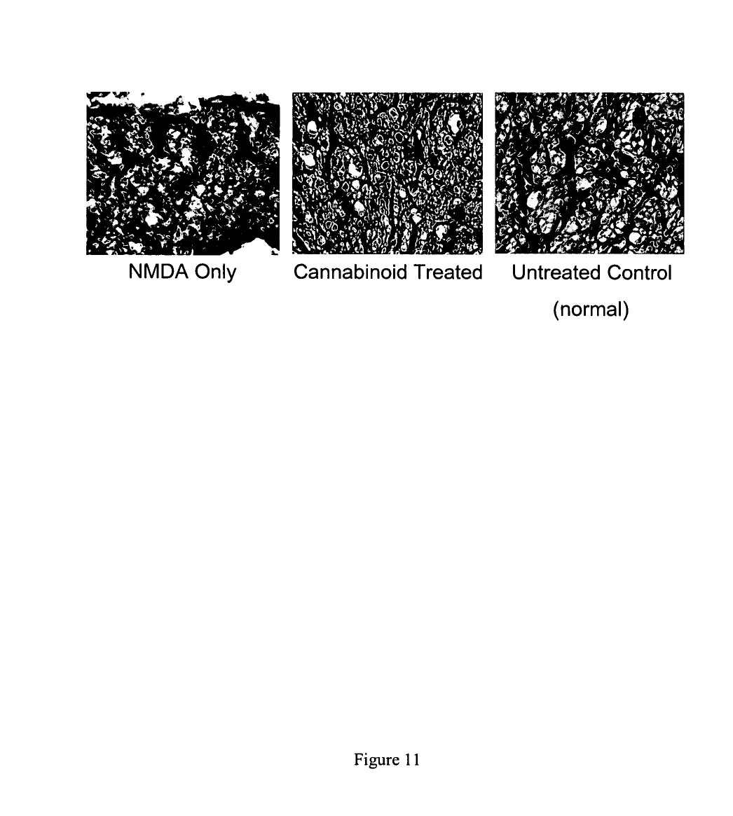

FIG. 12. Combination Therapy. Additive effects with cannabinoids and timolol were observed while synergistic effects with two synthetic cannabinoids were observed. Within 30 minutes, all combinations significantly decreased IOP. Unlike timolol alone, after the addition of the cannabinoids, IOP reduction was maintained for over 120 min. The combination of O-1812 and O-2545 had the greatest effect. No combination caused ocular irritation or systemic effects.

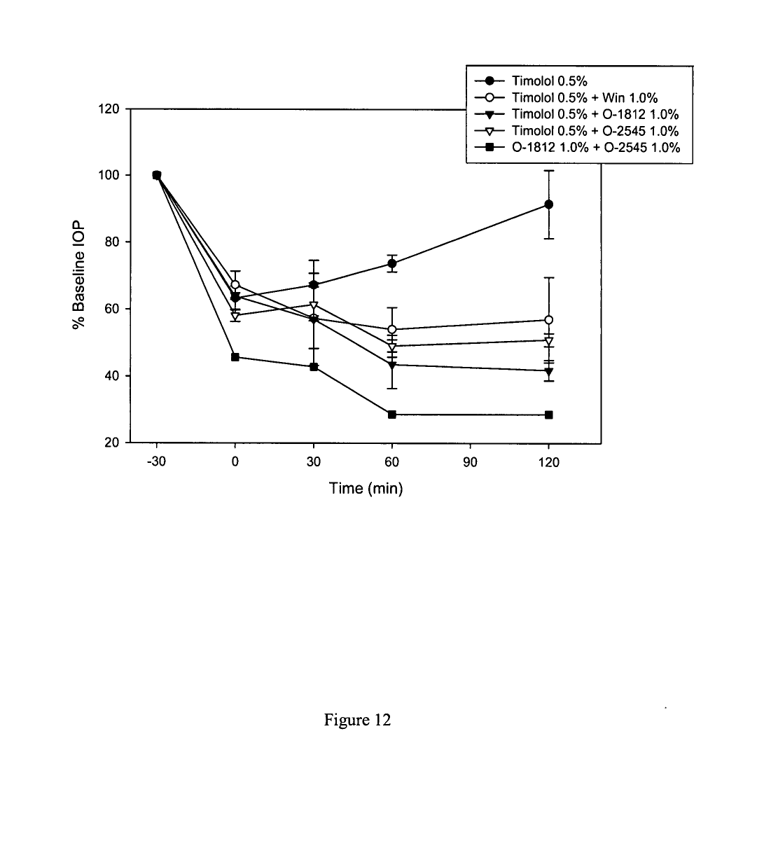

### DETAILED DESCRIPTION OF THE INVENTION

The invention provides water- and lipid-soluble cannabinoid compounds that are useful for the treatment of potential blinding diseases, such as glaucoma, diabetic retinopathy and macular degeneration. These conditions are all associated with degeneration of the retina and optic nerve. Elevated IOP is a major risk factor for glaucoma. There is also a significant number of glaucoma patients with normal IOP but increasing retinal damage. This argues for a direct component in glaucoma resulting in damage to the retina and optic nerve that is independent of IOP. It has been discovered that the compounds of the invention described herein provide unexpected beneficial results in both reducing IOP and protecting the retina.

In preferred embodiments, a topical administration route delivers the drugs. In certain embodiments, alternative administration routes may be employed, such as the direct injection of the drugs into the vitreous chamber, known as intravitreal injection. The efficacy of the drugs is similar and independent of the administration route. While glaucoma, diabetic retinopathy and macular degeneration are the most prevalent, other ocular degenerative diseases may be treated as well, such as Retinopathy of Prematurity (ROP), hypertensive retinopathy, age-related macular degeneration, UV-induced retinal damage and drug-related retinopathy. In further embodiments, these cannabinoids may be used to treat other neurodegenerative disorders such as Alexander disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease (Spinocerebellar ataxia type 3), Multiple sclerosis, Multiple System Atrophy, Neuroborreliosis, Parkinson disease, Pelizaeus-Merzbacher Disease, Pick's disease, Primary lateral sclerosis, Prion diseases, Refsum's disease, Sandhoff disease, Schilder's disease, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski diseaseand Tabes dorsalis.

The invention provides safe and effective methods of treating and /or delaying the progression of neuropathy in a patient in need thereof by administering a therapeutically effective amount of the compounds of the invention. “Therapeutically effective amount” refers to the amount of the compound and/or composition that is effective to achieve its intended purpose. “Neuropathy” refers to a disturbance in the function of a nerve or particular group of nerves. “Patient” refers to animals, preferably mammals, most preferably humans, and includes males and females, and children and adults. The compounds of the invention may be administered separately or may be administered in the form of one or more compositions that further comprise a carrier. The compounds of the invention may be administered daily to the patient or less frequently. The administration of pharmaceutical compositions of the present invention may be intermittent, or at a gradual, or continuous, constant or controlled rate to a warm-blooded animal. In addition, the time of day and the number of times per day that the pharmaceutical formulation is administered may vary.

In one embodiment, the invention provides compounds for daily administration or intake that comprise compounds in an amount from about 120 mg/day to about 6000 mg/day. In another embodiment, the invention provides a method that comprises topically administering to the eye a submicron emulsion containing the active compound that reduces eye irritation. This enables increased amounts of the drug to be administered without unacceptable irritation. In yet another embodiment, the invention provides a method for direct injection of the drugs into the vitreous chamber, or intravitreal injection. It has been unexpectedly discovered that compositions comprising combinations of the compounds of the invention may provide synergistic (i.e., greater than additive) effects in the treatment of IOP and retinal neuropathy.

The effective dose may vary, depending upon factors such as the condition, size and age of the patient, the severity of the symptoms being treated, and the manner in which the pharmaceutical composition is administered, as will be appreciated by the person of ordinary skill. For human patients, the dose of the compounds of the herein above described formulae may generally be administered in a dosage range amount of about 0.01 to about 100 mg/24 hr./patient, preferably about 0.1 to about 25 mg/24 hr./patient, and more preferably about 1 to about 10 mg/24 hr./patient. Preferably, the dosage may be divided up into several smaller dosages administered at intervals over each 24 hr. period and conventional extended release formulations may also be employed. The oral administration may be accomplished using conventional aqueous or non-aqueous pharmacological solutions, suspensions, emulsions, syrups, elixirs, and so forth, which have the active solubilized therein. Administration of the compounds of the invention may be determined and effected using conventional methods known in the art, as described, for example, in Remington: The Science and Practice of Pharmacy, University of the Sciences in Philadelphia. May 2005, 21 st Edition.

The compounds and/or compositions of the invention may be administered by any available and effective delivery system including, but not limited to, orally, bucally, parenterally, by inhalation spray, by topical application, by injection, transdermally, or rectally (e.g., by the use of suppositories) in dosage unit formulations containing conventional nontoxic pharmaceutically acceptable carriers, adjuvants, and vehicles, as desired. Parenteral includes subcutaneous injections, intravenous, intramuscular, intrasternal injection, or infusion techniques. Different compounds may be administered by different delivery systems and/or by different dosage forms.

Solid dosage forms for oral administration may include capsules, sustained-release capsules, tablets, sustained release tablets, chewable tablets, sublingual tablets, effervescent tablets, pills, powders, granules and gels. In such solid dosage forms, the active compounds may be admixed with at least one inert diluent such as sucrose, lactose or starch. Such dosage forms may also comprise, as in normal practice, additional substances other than inert diluents, e.g., lubricating agents such as magnesium stearate. In the case of capsules, tablets, effervescent tablets, and pills, the dosage forms may also comprise buffering agents. Soft gelatin capsules may be prepared to contain a mixture of the active compounds or compositions of the invention and vegetable oil. Hard gelatin capsules may contain granules of the active compound in combination with a solid, pulverulent carrier such as lactose, saccharose, sorbitol, mannitol, potato starch, corn starch, amylopectin, cellulose derivatives of gelatin. Tablets and pills may be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Injectable preparations, for example, sterile injectable aqueous or oleaginous suspensions may be formulated according to the known art using suitable dispersing agents, wetting agents and/or suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be used are water, Ringer's solution, and isotonic sodium chloride solution. Sterile fixed oils are also conventionally used as a solvent or suspending medium.

The bioavailabilty of the compositions may be enhanced by micronization of the formulations using conventional techniques such as grinding, milling, spray drying and the like in the presence of suitable excipients or agents such as phospholipids or surfactants.

The compounds and compositions of the invention may be formulated as salt forms. Salts include, for example, alkali metal salts and addition salts of free acids or free bases. Suitable acid addition salts may be prepared from an inorganic acid or from an organic acid. Examples of such inorganic acids include, but are not limited to, hydrochloric, hydrobromic, hydroiodic, nitric, carbonic, sulfuric and phosphoric acid and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, β-hydroxybutyric, cyclohexylaminosulfonic, galactaric and galacturonic acid and the like. Suitable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound.

While individual needs may vary, determination of optimal ranges for effective amounts of the compounds and/or compositions is within the skill of the art. Generally, the dosage required to provide an effective amount of the compounds and compositions, which may be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as the activity, efficacy, pharmacokinetic and toxicology profiles of the particular compound used, whether a drug delivery system is used, and whether the compound is administered as part of a drug combination.

### I. EXAMPLES

Materials and Methods

Rat Glaucoma Model. Compared to acute studies, a chronic rat model of glaucoma was generated. The rats were made glaucomatous by ligating three vortex veins (Hosseini, A. et al. (2006) Chronic topical administration of WIN-55-212-2 maintinas a reduction in IOP in a rat glaucoma model without adverse effects. Exp. Eye Res. 82(5):753-9). More specifically, ocular hypertension was induced in Sprague-Dawley rats by ligating 3 of 4 episcleral veins of the right eye using 9.0 nylon sutures under anesthesia with 3 mg/Kg acepromazine and 20 mg/Kg ketamine. Within 6 wk, IOP increased by at least 5 mm Hg in the operated eye. This intervention produced at least a 30% increase in IOP in the operated eye and lasted for up to 60 weeks. During this time, a 30% loss of retinal function was documented by ERG. Histological analysis of these retinas revealed a 40% loss of RGC. Thus, the model accurately mimics the progressive damage that occurs in patients with glaucoma.

Measurements of Intraocular Pressure. IOP elevation was measured under mild sedation using a calibrated Goldmann tonometer modified for rats. Three determinations were averaged for each eye. To account for diurnal variation, all IOP's were measured between 11 am and 1 pm during the daily trough period.

Heart Rate and Blood Pressure. BP and HR were measured with a tail cuff apparatus and the data was analyzed using Dasy Lab software (DasyTEC, Amherst, N.H.).

Therapeutic Regimen. For IOP Measurements (Topical application) Baseline IOP, heart rate, blood pressure and normal ocular anatomy were documented prior to drug administration. The drugs WIN 55-212-2 (0.1%, 1.0%), O-1812 (0.01%, 0.1%, 1.0%), O-2545 (0.1%, 1.0%) were all dissolved in Tocrisolve™ (vehicle). IOP, heart rate, and blood pressure were measured prior to and after 30, 60 and 120 min after topical administration. Contralateral eyes served as vehicle treated controls.

Slit Lamp Biomicroscopy/Digital Photography. Ocular toxicity was measured using slit lamp examination and performed using a modified MacDonald-Shadduck semi-quantitative scale for ocular irritation (McDonald, T. O. and Shadduck, J. A. (1987) Eye irriation. In Dermatotoxicology. 3rd Ed. Mazulli and Maibach. Pp 641-696. Washington, DC: Hemisphere). All eyes were examined by a slit lamp fitted with a color digital camera for digital photography of conjunctiva, cornea, and anterior camber. Images were stored and analyzed using a digital video system (Lombart Instruments DVS System). At baseline and following drug administration, eyes were examined for conjunctival congestion, conjunctival discharge, corneal edema, aqueous flare, fibrin, and iris congestion (Samudre, S. S. et al. (2004) Comparison of Topical Steroids for Acute Anterior Uveitis. J. Ocul. Pharmacol. Ther. 20 (6):533-547). Knowledgeable, masked observers rated each factor on a semi-quantitative four point scale where zero was coded normal, 1=minimal, 2=moderate and 3=severe involvement. Results were compared to the normal baseline evaluation performed before treatment and also with the untreated contralateral eye.

TABLE I
Ocular Toxicity: Comparison of Eyes Using the MacDonald-Shadduck
System for Scoring Ocular Irritation

| | Grade | | | | |
|---|---|---|---|---|---|
| Criteria | 0 | 1 | 2 | 3 | 4 |
| Conjunctival Congestion | X | | | | |
| Conjunctival Chemosis | X | | | | |
| Conjunctival Discharge | X | | | | |
| Aqueous Flare | X | | | | |
| Loss of Light Reflex | X | | | | |
| Iris Hyperemia | X | | | | |
| Corneal Haze | X | | | | |
| Corneal Irritation | | X | | | |
| Corneal Vascularization | X | | | | |

Grade 1 corneal irritation was observed in both eyes e.g. treated and untreated. This finding was most likely due to repeated application of the Goldmann tonometer.

TABLE II
IOP Reduction Profiles of all compounds at 60 min

| Drug | IOP after 60 min (% of Baseline) |
|---|---|
| O-1812 0.01% | 81.9 ± 2.7* |
| O-1812 0.10% | 78.3 ± 3.1* |
| O-1812 1.0% | 61.9 ± 3.9* |
| O-2545 0.10% | 77.8 ± 2.8* |
| O-2545 1.0% | 76.4 ± 2.8* |
| WIN 55-212-2 0.01% | 83.1 ± 5.5* |
| WIN 55-212-2 1.0% | 58.3 ± 5.6* |
| WIN 55-212-3 1.0% | 86.5 ± 2.5* |

*Significantly lower than baseline, p < 0.05. All synthetic cannabinoids decreased IOP and had no effect on BP or HR, unlike those changes associated with systemic administration. O-1812 1.0% and WIN 55-212-2 1.0% were most efficacious. The cornea, conjunctiva and anterior chamber were normal with no signs of ocular inflammation.

TABLE III
The binding affinity (means ± SE, N = 3) for CB1 and CB2 receptors are presented below. The ability of the
analogs to inhibit spontaneous activity (SA), to produce antinociception in the tail-flick procedure (TF) and
produce relative immobility (RI) is presented as ED50 values (mg/kg) below. These analogs produced
relatively little hypothermia (data not shown).

| | | CB1 KI | CB2 KI | S.A. | T.F. | R.I. |
|---|---|---|---|---|---|---|
| Name | Structure | (nM) | (nM) | ED50 mg/kg | ED50 mg/kg | ED50 mg/kg |
| O-2954 | 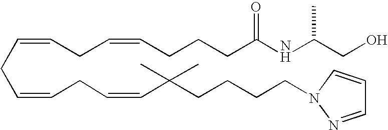 | 127 ± 2.84 | 301 ± 35.6 | 0.14 (0.10-0.20) | 0.19 (0.15-0.25) | 0.57 (0.52-0.62) |
| O-2955 | 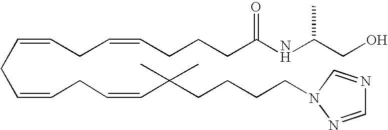 | 298 ± 33.9 | 215 ± 40.5 | 2.03 (1.38-2.98) | 1.94 (1.42-2.65) | 2.21 (1.68-2.91) |
| O-4316 | 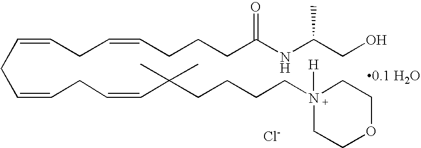 | 3.88 ± 0.22 | 22.4 ± 0.43 | 0.11 (0.06-0.18) | 0.04 (0.02-0.06) | 0.03 (0.02-0.05) |
| O-4368 | 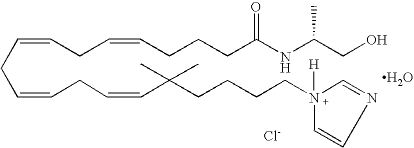 | 90.5 ± 5.0 | 153 ± 5 | 0.68 (0.34-1.37) | 0.29 (0.19-0.44) | 0.25 (0.15-0.41) |
| O-4386 | 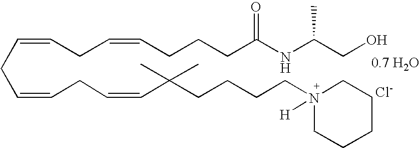 | 92.8 ± 2.2 | 226 ± 24 | 0.79 (0.52-1.18) | 0.55 (0.30-1.01) | 0.70 (0.43-1.12) |
| O-4388 | 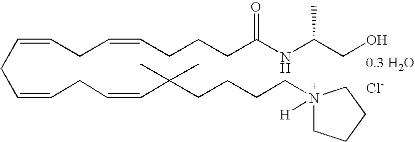 | 99.4 ± 5.9 | 404 ± 39 | 0.42 (0.28-0.65) | 0.38 (0.17-0.86) | 0.84 (0.66-1.08) |

TABLE IV
Combined effects of timolol and compounds of the invention on
IOP in a rat glaucoma model

| | IOP |
|---|---|
| Drug | at 120 min (% Baseline) |
| Timolol 0.5% | 91.5 ± 10.2 |
| WIN 1.0% | 48.8 ± 2.8* |
| O-1812 1.0% | 62.0 ± 0.5* |
| O-2545 1.0% | 65.5 ± 0.8* |
| Timolol 0.5%, then WIN 1.0% | 56.9 ± 12* |
| Timolol 0.5%, then O-1812 1.0% | 41.8 ± 3.1*† |
| Timolol 0.5%, then O-2545 1.0% | 50.9 ± 1.9*‡ |
| O-1812 1.0% + O-2545 1.0% | 41.1 ± 8.8*†‡ |

*significantly less than baseline, p < 0.05
†significantly less than O-1812 1.0% alone, p < 0.05
‡significantly less than O-2545 1.0% alone, p < 0.05

Confocal Microscopy. Repeated noninvasive in vivo visualization of cornea and anterior segments for the presence, location and number of inflammatory cells, as well as fibrin, hyper-refractile bodies and changes in epithelial, stromal and endothelial cell morphology was performed by confocal microscopy. Prior to applanation, rats were sedated and 0.5% proparacaine was applied topically. Data was analyzed using the Metamorph imaging system (Universal Imaging, Downingtown, Pa.).

Rat NMDA Model. To mimic glaucomatous damage to the retina without a potentially confounding effect on IOP, retinal damage was induced by NMDA in a rat model. Following baseline measurements, e.g. IOP, ERG, slit lamp, fundus photography, heart rate and blood pressure, N-methyl D-aspartate (NMDA, 2 μl of 10 mM) was injected intravitreally to induce RGC damage. For identification purposes, each rat had a subcutaneous microchip in place. For positive identification, a hand scanner was used each time the rat was examined. This was necessary since the examiners will be masked as to treatment group. Treatment groups consisted of either NMDA only (control), O-1812 (0.1%) or O-2545 (0.1%), O-1812 (0.1%)+SR-141716 (2 mM), O-2545 (0.1%)+SR-141716 (2 mM), O-1812 (0.1%)+SR-144258 (2 mM) and O-2545 (0.1%)+SR-144258 (2 mM). All experiments were conducted in accordance with the policies of the Institutional Animal Care and Use Committee and the ARVO policy on use of animals in experiments. A minimum of two baseline ERG measurements was recorded 1 wk prior, 1 wk and 2 wk post injection. At the end of each experiment, rats were sacrificed and their eyes were preserved for histological assessment of effects on the retina and optic nerve.

Electroretinogram. In normal retina, full field electroretinography (ERG) stimulates both rods (a-waves) and cones (b-waves). A decrease in their amplitudes indicates retinal functional damage. Scotopic ERG changes were measured in rats that were dark-adapted for at least 4 hours. Eyes were then dilated with 1% atropine after which 0.5% proparacaine and methylcellulose gel (GPS 2.5%) was applied topically. Custom made AgCl electrodes were placed on the apex of the cornea. Stimuli consisted of 10-μsec flashes of unattenuated white light generated by a Ganzfeld bowl photo stimulator. Data from each eye were recorded separately with a driver amplifier and acquired digitally via DASYLab. The contralateral normal eye served as an age matched negative control, e.g. response in a normal, undamaged eye. Differences in amplitude of the a- and b-waves between the operated eye and the contralateral eye were calculated and analyzed by ANOVA and t-test.

Histology. For retinal flat mounts, retinas were dissected from formalin fixed whole eyes, mounted on superfrost glass slides, stained with hematoxylin and eosin and digitally photographed. For analysis, the retina was divided into four quadrants. A minimum of five 50 μm² sections per quadrant were used to count cell number. The cell sections were selected to be equidistant from the optic cup for all quadrants. For optic nerve experiments, the optic nerve was dissected at the margin of the optic disk and embedded in cryo media. Sections of 10 μm thickness were cut and mounted on superfrost glass slides and stained with hematoxylin and eosin. The slides were digitally photographed.

ICR Mice and Materials. Male ICR mice (Harlan Laboratories, Indianapolis, Ind.) weighing between 24 to 30 g were used in all experiments. Mice were maintained on a 14:10-hr light/dark cycle with food and water available ad lib. All test groups consisted of 6 to 12 mice. Analogs dissolved in a vehicle consisting of ethanol, emulphor and saline in a ratio of 1:1:18. All chemicals for receptor binding studies were purchased from Sigma (St. Louis, Mo.) except the following: Dulbeco's modified Eagle's medium (DMEM) from GIBCO BRL (Grand Island, N.Y.), Whatman GF/B glass fiber filters from Fischer Scientific (Pittsburg, Pa.), fetal calf serum (FCS) and fetal bovine serum (FBS) from HyClone Laboratories (Logan, Utah) and Budget-Solve scintillation fluid from RPI Corp. (Mount Prospect, Ill.).

Membrane Preparations. HEK-293 cells stably expressing the human CB₁ receptor were cultured in DMEM with 10% FBS and Chinese Hamster Ovary (CHO) cells stably expressing the human CB₂ receptor were cultured in DMEM with 10% FCS. Cells were harvested by replacement of the media with cold phosphate-buffered saline containing 1 mM EDTA followed by centrifugation at 1000×g for 5 min at 4° C. The pellet was resuspended in 50 mM Tris-HCl containing 320 mM sucrose, 2 mM EDTA and 5 mM MgCl2 (pH 7.4) (centrifugation buffer), then centrifuged at 1000×g for 10 min at 4° C. and the resulting supernatant was saved. This process was repeated twice. The supernatant fractions were combined and centrifuged at 40,000×g for 30 min at 4° C. The resulting P2 pellet was resuspended in assay buffer (50 mM Tris-HCl (pH 7.4), 3 mM MgCl₂, 0.2 mM EGTA, and 100 mM NaCl) and protein was measured. Membranes were stored at −80° C. until use.

Receptor Binding. Membrane homogenates (50 μg) were incubated with 0.5 nM [³H]CP55,940 in the presence of varying concentrations (1 nM-10 μM) of test compounds in 0.5 ml of buffer containing BSA (5 mg/ml). Non-specific binding was measured in the presence of 1 μM CP55,940. The assay was incubated at 30° C. for 1 hr and terminated by addition of ice cold 50 mM Tris-HCl+BSA (1 mg/ml) (pH 7.4) followed by filtration under vacuum through Whatman GF/B glass fiber filters with 3 washes with cold Tris buffer. Bound radioactivity was determined by liquid scintillation spectrophotometry at 50% efficiency after extraction by shaking samples for 30-60 min with Budget-Solve scintillation fluid. Data are reported as the mean ±SEM of three experiments, each performed in triplicate. Kᵢ values were calculated from displacement data using EBDA (Equilibrium Binding Data Analysis; BIOSOFT, Milltown, N.J.).

Behavioral Evaluations. All animals were allowed to acclimate to the observation room overnight. Behavioral effects were assessed in the tetrad model to measure potency in decreasing spontaneous activity (SA), antinociception [tail-flick procedure, TF], rectal temperature (RT) and relative immobility (RI) catalepsy. The baselines for tail-flick latency (2-4 sec) and rectal temperature were determined prior to i.v. injections. Baseline rectal temperatures were measured using a telethermometer and a thermometer probe inserted to 25 mm (Yellow Springs Instrument Co., Yellow Springs, Ohio). The mice treated i.v. with an analog were placed in individual photocell activity chambers 5 min later. Spontaneous activity wasp monitored for 10 min in a Digiscan Animal Activity Monitor (Omnitech Electronice, Inc., Columbus, Ohio) as measured by the number of interruptions of 16 photocell beams per chamber. The total number of beam interruptions during the 10-min period was determined and presented as total counts. The mice were then assessed at 20 min following the i.v. injection for antinociception using the tail-flick reaction time to a heat stimulus. A 10-sec maximum latency was used in order to avoid tail injury. The results are presented as % MPE and are calculated as follows: % MPE=[(test latency-control latency)/(10 sec−control latency)]×100.

Rectal temperature was measured 30 min after the i.v. injection. The change in rectal temperature (Δ° C.) following analog administration was calculated for each animal. Relative immobility (catalepsy) was measured 40 min after the i.v. injection by the ring-immobility test. Mice were placed on a ring 5.5 cm in diameter attached to a stand at a height of 16 cm. The amount of time the mice spent motionless on the ring during the 5-minute procedure was measured, with the criteria of immobility being defined as the absence of all voluntary movements, including whisker movement, but excluding respiration. The percent immobility was calculated as: % immobility=[time immobile (sec)]/[length of session (sec)]×100. Mice that fell from the ring or actively jumped were allowed five attempts. After the fifth escape these mice were removed from the ring and not included in the calculations. Data were collected from 6-12 mice for each condition tested.

A similar protocol was used to determine the effects of analogs following either i.t. or i.c.v. injection. The method of Hylden and Wilcox (Hylden and Wilcox, 1980) was used to inject 5 μl of solution i.t. between L5 and L6 of the spinal cord with a 30-gauge needle. I.c.v. injections were performed as described earlier (Pedigo et al., 1975). Mice were anesthetized with 2.5% isoflurane, and a transverse incision was made in the scalp. A free-hand 5 ml injection of drug was made into the lateral ventricle. Mice were tested as described above in the activity champers 5 to 15 min after the injection. Antinociception, body temperature, and catalepsy were quantitated 20, 20 and 40 min, respectively, after the injection.

All studies were carried out in accordance with the Declaration of Helsinki and Guide for the Care and Use of Laboratory Animals as adopted and promulgated by the U.S. National Institutes of Health.

Data analysis. Based on data obtained from numerous previous in vivo studies with cannabinoids, maximal cannabinoid effects in each in vivo pharmacological procedure were estimated as follows: 90% inhibition of spontaneous activity, 100% MPE in the tail flick procedure, and −6° C. change in rectal temperature. Means and standard error (S.E.) were calculated for % MPE, number of photocell disruptions, % ring immobility and Δ° C. Analysis of variance (ANOVA) was used to determine significant differences between control and treatment groups followed by Dunnett's t-test post-hoc analysis. Statistical analysis was performed using StatView, version 5.0 (SAS Institute, Cary, N.C.). Significance was defined as a “p”<0.05. ED₅₀'s were defined as the dose at which half maximal effect occurred. For drugs that produced one or more cannabinoid effect, ED₅₀'s were calculated separately using least-squares linear regression on the linear part of the dose-effect curve for each measure in the mouse tetrad, plotted against log₁₀ transformation of the dose.

Combination Therapy. Since cannabinoids and timolol, which are both known to reduce IOP, have different mechanisms of action, there is potential for a synergistic effect. The combination of timolol and a synthetic cannabinoid (WIN55 212-2, O-1812, or O-2545) to reduce IOP in a chronic rat model of ocular hypertension was examined. Surgical ligation of three of the four episcleral veins in one eye of Sprague Dawley rats caused a long lasting (>44 week) increase in IOP. IOP was measured by Goldmann tonometry at baseline (−30), 0, 30, 60 and 120 min, as was heart rate and blood pressure. For combination therapy, after baseline IOP measurement, timolol 0.5% was applied topically followed 30 min later by WIN55 212-2 1.0%, O-1812 1.0%, or O-2545 1.0%. In another experiment, O-1812 1.0% and O-2545 1.0% were administered simultaneously. An analysis for ocular irritation was performed by slit lamp examination (SLE) at baseline and 150 min. In prior experiments timolol significantly decreased IOP for only 30 min in this model.

### II. RESULTS

In an ocular hypertensive model, topically applied WIN 55-212-2 and O-1812 are effective, non-toxic ocular hypotensive agents. Lipid soluble O-1812 had the most rapid onset, but the more water soluble O-2545 had the longest duration of action. Based upon their pharmacokinetic characteristics, the novel eicosanoids listed in Table III are expected to be as efficacious, if not more potent, than O-1812 and O-2545.

Compared to timolol alone, combination therapy with timolol and cannabinoids prolonged both the duration and magnitude of their effect on IOP. However, the combination of two synthetic cannabinoids also had synergistic effects. The potential for multidrug therapy using cannabinoids may provide a greater benefit.

Although the invention has been set forth in detail, one skilled in the art will appreciate that numerous changes and modifications may be made to the invention, and that such changes and modifications may be made without departing from the spirit and scope of the invention.

We claim:

1. A compound having the formula (I): 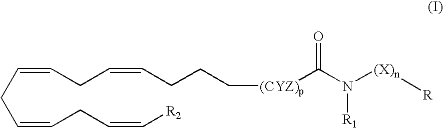
wherein
R is CH₃, OH, Cl, F, Br, I, or CF₃;
R₁ is H, or C₁-C₆ alkyl;
X is alkyl, branched alkyl, cyclopropyl, or cycloalkyl;
Y is H, alkyl, or branched alkyl;
Z is H, alkyl, or branched alkyl;
p is 0-4; and
R₂ is 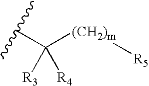
wherein
R₃ is H, alkyl, or branched alkyl;
R₄ is H, alkyl, or branched alkyl;
m is 1-7; and
R₅ is CH₃, OH, Cl, Br, I, CN, ONO, ONO₂, NO₂, pyrazole, triazole, morpholine, piperidine, pyrrolidine, or imidazole;
provided that when R is OH, R₁ is H, X is isopropyl, p is 0, R₃ is methyl, R₄ is methyl, m is 4, R₅ cannot be CN.

2. The compound of claim 1 selected from the group consisting of:

| Compound | Structure |
|---|---|
| O-2954 | 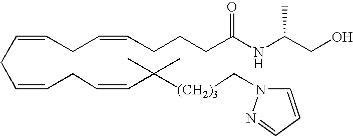 |
| O-2955 | 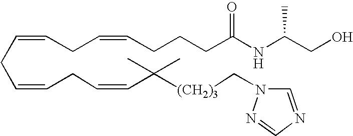 |
| O-4316 | 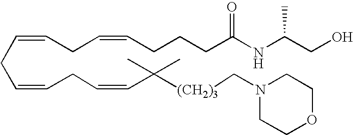 |
| O-4368 | 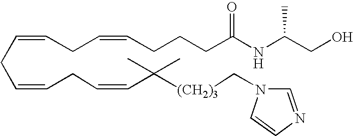 |

and

| | |
|---|---|
| O-4386 | 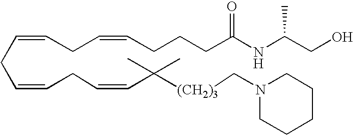 |

3. A method for protecting a patient against a disorder characterized by damage to the retina or optic nerve, comprising administering to a patient in need thereof an effective amount of a compound, or pharmaceutically acceptable salt thereof, having the formula (I): 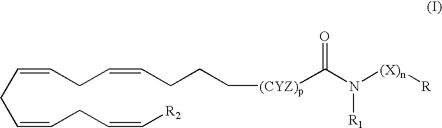
wherein
R is CH₃, OH, Cl, F, Br, I, or CF₃;
R₁ is H, or C₁-C₆ alkyl;
X is alkyl, branched alkyl, cyclopropyl, or cycloalkyl;
Y is H, alkyl, or branched alkyl;
Z is H, alkyl, or branched alkyl;
p is 0-4; and
R₂ is 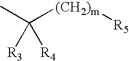
wherein
R₃ is H, alkyl, or branched alkyl;
R₄ is H, alkyl, or branched alkyl;
m is 1-7; and
R₅ is CH₃, OH, Cl, Br, I, CN, ONO, ONO₂, NO₂, pyrazole, triazole, morpholine, piperidine, pyrrolidine, or imidazole.

4. The method of claim 3 wherein the compound is selected from the group consisting of:

| Compound | Structure |
|---|---|
| O-2954 | 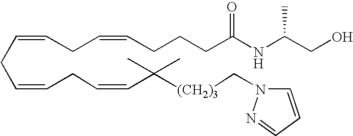 |
| O-2955 | 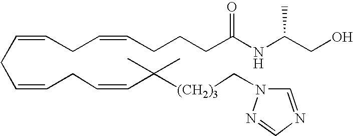 |
| O-4316 | 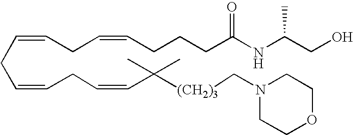 |
| O-4368 | 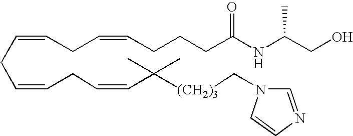 |
| O-4386 | 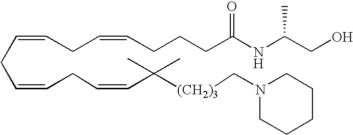 |
| O-4388 | 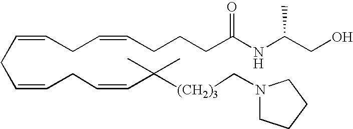 |

and

| | |
|---|---|
| O-1812 | 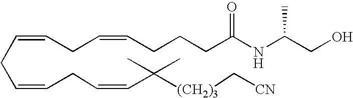 |

5. A method for protecting a patient against a disorder characterized by damage to the retina or optic nerve, comprising administering to a patient in need thereof an effective amount of a compound, or pharmaceutically acceptable salt thereof, having the formula (II): 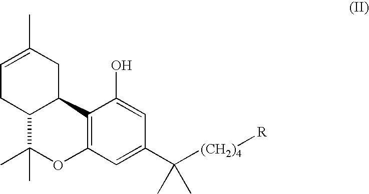
wherein R is imidazole, pyrazole, triazole, or morpholine.

6. The method of claim 5 wherein the compound is selected from the group consisting of:

| Compound | Structure |
|---|---|
| O-2545 | 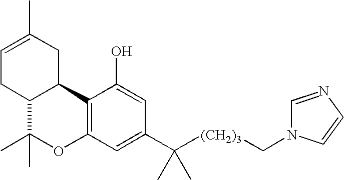 |
| O-2651 | 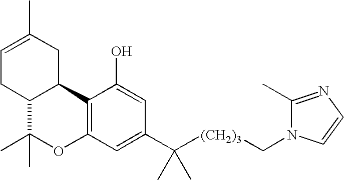 |
| O-2715 | 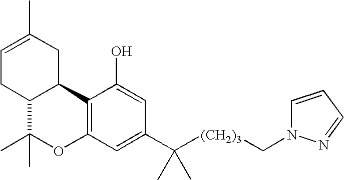 |
| O-2716 | 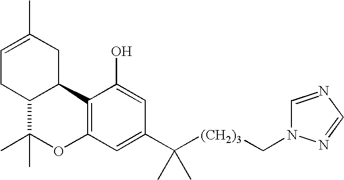 |
| O-2737 | 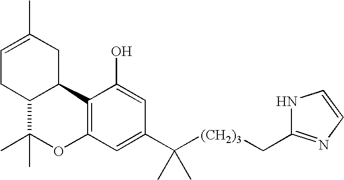 |

and

| | |
|---|---|
| O-3226 | 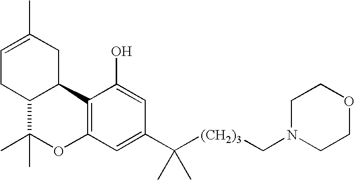 |

7. A method for protecting retinal ganglionic cells, comprising administering an effective amount of a compound, or pharmaceutically acceptable salt thereof, having the formula (I): 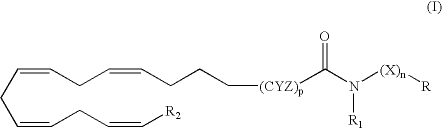
wherein
R is CH₃, OH, Cl, F, Br, I, or CF₃;
R₁ is H, or C₁-C₆ alkyl;
X is alkyl, branched alkyl, cyclopropyl, or cycloalkyl;
Y is H, alkyl, or branched alkyl;
Z is H, alkyl, or branched alkyl;
p is 0-4; and
R₂ is 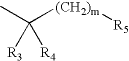
wherein
R₃ is H, alkyl, or branched alkyl;
R₄ is H, alkyl, or branched alkyl;
m is 1-7; and
R₅ is CH₃, OH, Cl, Br, I, CN, ONO, ONO₂, NO₂, pyrazole, triazole, morpholine, piperidine, pyrrolidine, or imidazole.

8. The method of claim 7, wherein the compound is selected from the group consisting of:

| Compound | Structure |
|---|---|
| O-2954 | 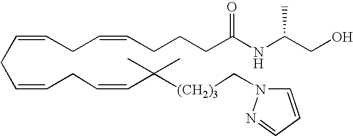 |
| O-2955 | 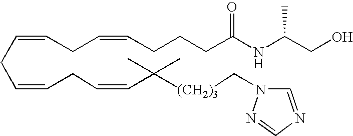 |
| O-4316 | 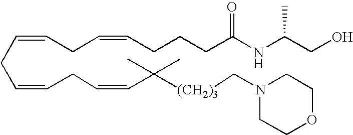 |
| O-4368 | 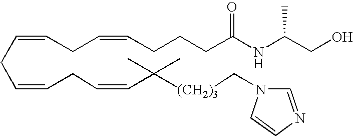 |
| O-4386 | 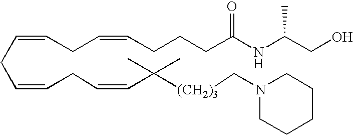 |
| O-4388 | 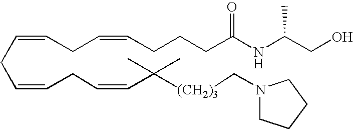 |

and

| | |
|---|---|
| O-1812 | 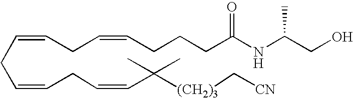 |

9. A method for protecting retinal ganglionic cells, comprising administering an effective amount of a compound, or pharmaceutically acceptable salt thereof, having the formula: having the formula (II): 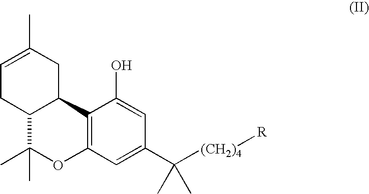
wherein R is imidazole, pyrazole, triazole, or morpholine.

10. The method of claim 9, wherein the compound is selected from the group consisting of:

| Compound | Structure |
|---|---|
| O-2545 | 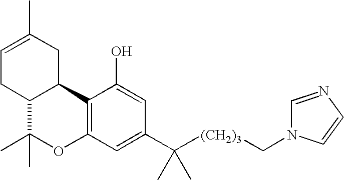 |
| O-2651 | 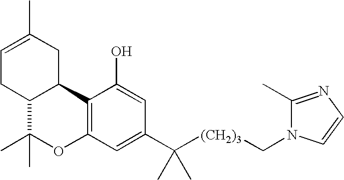 |
| O-2715 | 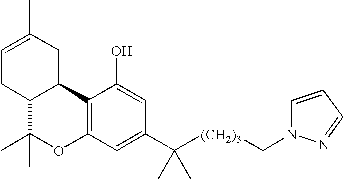 |
| O-2716 | 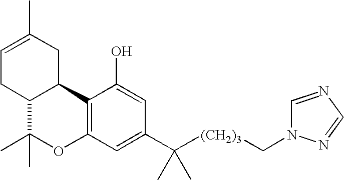 |
| O-2737 | 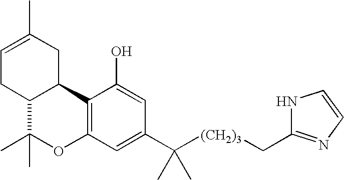 |

and

| | |
|---|---|
| O-3226 | 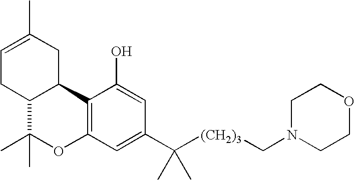 |

11. The method of claim 7 or 9, wherein said retinal ganglionic cells are in vivo.
